(19) United States
(12) Patent Application Publication
(10) Pub. No.: US 2007/0123717 A1
(43) Pub. Date: May 31, 2007

(54) Method for the production of 2-keto-l-gulonic acid esters

(75) Inventors: Thomas Domschke, Speyer (DE); Martin Merger, Frankenthal (DE); Frank Haese, Bollingstedt (DE); Peter Resch, Hettenleidelheim (DE); Tillmann Faust, Weisenheim (DE)

Correspondence Address: CONNOLLY BOVE LODGE & HUTZ, LLP, P O BOX 2207, WILMINGTON, DE, 19899, US

(73) Assignee: Basf Aktiengesellschaft, Ludwigshafen (DE)

(21) Appl. No.: 10/582,911

(22) PCT Filed: Dec. 10, 2004
(86) PCT No.: PCT/EP04/14069

§ 371 Date: Jun. 14, 2006

(30) Foreign Application Priority Data
Dec. 15, 2003 (DE) 10359023.4

(51) Int. Cl. C07D 307/62

(52) U.S. Cl. 549/314

(57) ABSTRACT

A method for the production of C₁-C₁₀-alkyl 2-keto-L-gulonates by esterification of 2-keto-L-gulonic acid anhydrate with an anhydrous C₁-C₁₀-alkyl alcohol in the presence of an acidic homogeneous catalyst in a reactor cascade comprising at least two reactors, one of these reactors being a tubular reactor, without the water forming in the esterification being removed from the reaction space.

The invention relates to a novel process for the production of 2-keto-L-gulonic acid esters. These esters are important intermediates for the synthesis of L-ascorbic acid (vitamin C).

The esterification of 2-keto-L-gulonic acid with an alcohol, in particular with a lower alkyl alcohol, under acidic catalysis is known from numerous publications. Such an esterification is usually effected in the presence of an acidic catalyst, e.g. sulfuric acid, hydrochloric acid, sulfonic acids or strongly acidic ion exchange resins.

The conversion to the ester is an equilibrium reaction with formation of water. The ketogulonic acid ester conversions and yields therefore depend to a great extent on the water content of the reaction mixture.

Frequently, the 2-keto-L-gulonic acid is used in the form of the monohydrate, i.e. with an equimolar proportion of water (WO 99/03853; U.S. Pat. No. 5,128,487).

In EP 0535927 A1, the water formed in the esterification from 2-keto-L-gulonic acid and alkyl alcohol is distilled off together with a part of the alcohol used and is replaced by fresh alcohol (as vapor).

The removal of water with the vapor is not very efficient in the case of lower alkyl alcohols having chain lengths of 1-3 carbon atoms since the alcohol fraction in the vapor phase is as a rule below 5%. Accordingly, a very large amount of alcohol has to be distilled off with high energy input; for example, 10 kg of methanol per kg of 2-keto-L-gulonic acid to be esterified. Moreover, this also requires a long residence time in the reaction space (up to 10 hours in the case of batch operation in a stirred container), which can lead to irreversible secondary reactions and decomposition of starting material and product.

WO 99/03853 describes a variant where a two-stage esterification is carried out. First, heating is effected without water, alcohol and water are then substantially evaporated off and esterification is then continued with fresh anhydrous alcohol.

JP-A 3-38579 and U.S. Pat. No. 5,128,487 describe the production of a pure 2-keto-L-gulonic acid ester also with initially incomplete esterification. The still free 2-keto-L-gulonic acid is converted selectively with a base into its salt and is then precipitated. This process is uneconomical since the ketogulonic acid salt not only has to be separated from the ester solution but also has to be protonated again (for example by ion exchange) and then has to be isolated by crystallization. Moreover, recovery of ester adhering to the ketogulonic acid salt is also necessary.

EP 0671405 describes the continuous production of 2-keto-L-gulonic acid esters, especially methyl and ethyl 2-keto-L-gulonate, by reacting 2-keto-L-gulonic acid and methanol or ethanol, respectively, in a tubular reactor which is filled with ion exchanger as acidic catalyst. 2-Keto-L-gulonic acid conversions of more than 98% are achieved there. A high initial methanol/ketogulonic acid ratio is necessary for this procedure in order to dissolve the ketogulonic acid completely and to ensure high conversions, since no water is removed during the esterification. In the mixture, the proportion of ketogulonic acid in methanol is 8-15% by weight, which corresponds to a weight ratio of 5.7-11.5:1. After the esterification, the solution has to be concentrated, i.e. a large part of the alcohol has to be removed, so that the losses of sodium ascorbate via removal by the mother liquor remain as small as possible in the subsequent lactonization to give the ascorbate. Moreover, the ion exchangers have only a limited life, since in particular polyhydroxy compounds rapidly occupy their surface.

DE 199 38980 describes a further continuous method for the production of ketogulonic acid esters. Ketogulonic acid is esterified in a liquid film on a hot surface with removal of water. However, removal of water is efficient only in the case of higher alcohols (e.g. n-butanol). According to the examples, the ketogulonic acid is even dissolved in water before being passed over the hot surface. Owing to the low esterification rate in the case of higher alcohols (from C₄) esterification must be effected at temperatures above 85° C. in order to keep the dimension of the apparatuses within economically expedient limits. At temperatures above 70° C., however, undesired discolorations of the end product occur.

It was therefore the object to provide a method for the production of C₁-C₁₀-alkyl 2-keto-L-gulonates which manage this without removal of the water formed and without the limitation of the life of a heterogeneous catalyst and nevertheless ensures a high conversion in an economical manner. Furthermore, the disadvantages mentioned at the outset in the prior art should be avoided.

A method for the production of C₁-C₁₀-alkyl 2-keto-L-gulonates by esterification of 2-keto-L-gulonic acid anhydrate with an anhydrous C₁-C₁₀-alkyl alcohol in the presence of an acidic homogeneous catalyst in a reactor cascade comprising at least two reactors, one of these reactors being a tubular reactor, without water forming in the esterification being removed from the reaction space has been found.

All customary alcohols having a chain length of from 1 to 10 carbon atoms, but in particular methanol, ethanol, n-propanol, isopropanol, n-butanol, isobutanol and tert-butanol, are suitable as the alkyl alcohol.

Anhydrous alcohols are defined as those alcohols which comprise less than 1% by weight, preferably less than 0.5% by weight and particularly preferably less than 0.2% by weight of water. Such anhydrous alcohols are commercially available or can easily be prepared by drying methods known to the person skilled in the art, for example molecular sieves.

The weight ratio of alkyl alcohol to 2-keto-L-gulonic acid can be varied within wide ranges. Weight ratios of from 1.5:1 to 5:1 (alkyl alcohol:2-keto-L-gulonic acid anhydrate) are preferably employed. In this instance, the ketogulonic acid must not be completely soluble in the alkyl alcohol. A very advantageous weight ratio, in particular for the esterification with methanol, is 3:1 since firstly sufficient ester solubility is achieved here and a high equilibrium conversion can be obtained (97 mol % of ester) and secondly concentration of the resulting ester solution before the further processing (alkaline lactonization) is avoided.

The alkyl alcohol required for the esterification simultaneously forms the solvent for the reaction. A further solvent can be used but is as a rule unnecessary.

A strong mineral acid, preferably sulfuric acid or hydrochloric acid, or strong organic acids, such as sulfonic acids, can be used as the homogeneous catalyst. The weight ratio of catalyst to ketogulonic acid depends on the strength of the acid used; in the case of sulfuric acid, it is, for example, 0.001-0.05:1.

The esterification is preferably carried out at atmospheric pressure and temperatures of from 50 to 70, in particular from 55 to 68° C. Higher pressures and temperatures result in the equilibrium being established more rapidly but also in undesired secondary reactions.

Pressure and temperature may differ in the individual cascades.

At least two reactors, preferably three reactors, are used as the reactor cascade, one of these reactors being a tubular reactor. Usually, the tubular reactor is connected as the last reactor in the cascade. The design/dimensions of the tubular reactor is familiar to the person skilled in the art and can be optimized for the method according to the invention on the basis of simple investigations. Further embodiments are dislosed in the experimental section. The tubular reactors may also comprise conventional packings in order to achieve thorough mixing of the reactants.

The average residence time in the reactors is cumulatively about 30 minutes to 10 hours, preferably 1-5 hours.

In the method according to the invention, the water forming in the esterification reaction is not removed from the reaction space, either selectively or together with the alkyl alcohol. This procedure is particularly economical because energy costs which are usually incurred by the removal of water/alcohol (distillation) can thus be saved.

After the end of the esterification, the catalyst acid can be neutralized with an appropriate amount of base.

The method according to the invention can be operated both batchwise and continuously. A preferred embodiment is the continuous procedure.

The 2-keto-L-gulonic acid ester obtained by the method according to the invention can be used with a base, preferably sodium hydroxide or sodium carbonate, directly for lactonization to give the corresponding ascorbate. Usually, a yield of 92-95% in combination with a purity of up to 96% is obtained.

Further developments of the method according to the invention are described in the subclaims.

### EXAMPLE 1

Cascade of 2 stirred reactors and one tubular reactor

The reactor cascade consisted of
1. 2.5 l stirred container with mass flow-controlled metering of crystalline 2-keto-L-gulonic acid anhydrate, methanol and sulfuric acid, pumped circulation for flushing the 2-keto-L-gulonic acid into a funnel, propeller stirrer and baffles, overflow to the next reactor (reactor volume used about 2 l);
2. 2.5 l stirred container with propeller stirrer and baffles, bottom dicharge to the next reactor with pump (reactor volume used about 2 l);
3. tubular reactor (tube coil 10 mm×1.98 mm×36 m, volume about 1 l).

Operation of the Esterification:

650 g/h of 2-keto-L-gulonic acid anhydrate (about 99% pure, water conent <0.5% by weight), 1950 g/h of methanol and 7.7 g/h of concentrated sulfuric acid were metered into the first reactor. The reactors were operated at atmospheric pressure and an internal temperature of about 65-66° C. with an average residence time of about 2 hours altogether.

The discharge comprised methyl 2-keto-L-gulonate in 96-97 mol % yield (determination by HPLC).

### EXAMPLE 2

Cascade of Two Stirred Containers and One Tubular Reactor

The cascade consisted of two 1 l stirred containers with pumped circulation and stirrer and a tubular reactor operated by the liquid phase method (1 l glass tube filled with glass balls, usable volume about 0.5 l) and having an overflow.

Operation of the Esterification:

200 g/h of 2-keto-L-gulonic acid anhydrate (about 99% pure, water content <0.5% by weight), 600 g/h of methanol and 2.3 g/h of concentrated sulfuric acid were metered into the first reactor. The reactors were operated at atmospheric pressure and an internal temperature of about 65-66° C. with an average residence time of about 2.5 hours altogether.

A yield of as much as 90-92 mol % of methyl 2-keto-L-gulonate was achieved at the outlet of the second stirred reactor. The discharge of the tubular reactor comprised methyl 2-keto-L-gulonate in 95-97% yield (determination by means of HPLC).

1. A method for the production of C₁-C₁₀-alkyl 2-keto-L-gulonates by esterification of 2-keto-L-gulonic acid anhydrate with an anhydrous C₁-C₁₀-alkyl alcohol in the presence of an acidic homogeneous catalyst in a reaction cascade comprising at least two reactors, one reactor being a tubular reactor, wherein water forming in the esterification is not removed in the reaction cascade.

2. The method according to claim 1, wherein methyl or ethyl 2-keto-L-gulonate is prepared.

3. The method according to claim 1, which is carried out continuously.

4. The method according to claim 1, wherein the weight ratio of alkyl alcohol to ketogulonic acid anhydrate is from 1.5:1 to 5:1.

5. The method according to claim 1, wherein the esterification is carried out at from 50 to 70° C. at atmospheric pressure.

6. The method according to claim 1, wherein the C₁-C₁₀-alkyl alcohol is methanol thereby producing methyl-2-keto-L-gulonate.

7. The method according to claim 1, wherein the C₁-C₁₀-alkyl alcohol is ethanol thereby producing ethyl-2-keto-L-gulonate.

8. The method according to claim 1, wherein the acidic catalyst is sulfuric acid, and the weight ratio of acid to ketogulonic acid is from 0.001:1 to 0.05:1.

9. The method according to claim 4, wherein the weight ratio is about 3:1.

10. The method according to claim 1, wherein the tubular reactor includes glass balls in the reaction volume.
